(19) United States
(12) Patent Application Publication
(10) Pub. No.: US 2018/0085295 A1
(43) Pub. Date: Mar. 29, 2018

(54) Hair Growth Composition

(71) Applicant: Devakinanda Pasupuleti, Grand Blanc, MI (US)

(72) Inventor: Devakinanda Pasupuleti, Grand Blanc, MI (US)

(21) Appl. No.: 15/717,156

(22) Filed: Sep. 27, 2017

Related U.S. Application Data
(60) Provisional application No. 62/400,676, filed on Sep. 28, 2016.

(51) Int. Cl. A61K 8/49; A61K 8/63; A61K 8/92; A61Q 7/00

(52) U.S. Cl. CPC A61K 8/4953; A61Q 7/00; A61K 8/922; A61K 8/63

(57) ABSTRACT

A hair growth composition is provided. The novel hair growth composition is configured to synergistically treat hair loss by promoting hair growth in individuals suffering from androgenetic alopecia, or “common baldness.” The hair growth composition includes 40 mg-200 mg of minoxidil, 0.2 mg-1 mg of finasteride, 70 mg-80 mg of amla oil, and 1000 mg-14,000 mg of coconut oil. This specific combination and concentration of ingredients is configured to provide the most synergistic, highly efficacious, and rapid effect, inducing the maximum amount of hair growth activity in individuals suffering from androgenetic alopecia.

### CROSS REFERENCE TO RELATED APPLICATIONS

This application claims the benefit of U.S. Provisional Application No. 62/400,676 filed on Sep. 28, 2016. The above identified patent application is herein incorporated by reference in its entirety to provide continuity of disclosure.

### BACKGROUND OF THE INVENTION

The present invention relates to compositions for treating hair loss and triggering hair growth. More specifically, the present invention relates to a composition including minoxidil, finasteride, amla oil, and coconut oil that promotes hair growth in men and woman having androgenetic alopecia.

In humans, each hair follicle goes through repeated cyclical periods of growth including an active growth stage (anagen), which can persist for approximately 2 to 6 years; a transition phase (catagen), which lasts for only a week or two; and a resting period (telogen), which lasts 3 to 4 months. The hair is shed at the end of the telogen phase, and a new hair is grown as the cycle repeats. In the human scalp, which contains approximately 100,000 hair follicles, normally about 86% of hair follicles are in anagen, 1% of hair follicles are in catagen and 13% of hair follicles are in telogen. Therefore, in a normal human adult, approximately 100 hairs are shed from the scalp per day.

Excessive hair loss, or alopecia, may be classified as being one of two types, either non-scarring alopecia and scarring alopecia, and can be caused by a wide variety of factors. For example, non-scarring alopecia has been attributed to genetics and advanced age; administration of drugs such as anti-cancer chemotherapeutic drugs and contraceptives; topical use of chemical treatments, such as hair dyes, permanent wave solutions, and straighteners; diseases, such as leprosy or syphilis; illness; allergy; and hair follicle infection. Scarring alopecia may be a consequence of burns, such as accidental or post-surgical burns from cryosurgery or laser surgery, or other types of trauma, which often causes destruction of follicles.

The most common type of human hair loss is androgenetic alopecia (also known as androgenic alopecia), which is a non-scarring hair loss of telogen hairs caused by an excessive androgen effect in genetically susceptible men and women. Androgens trigger the miniaturization or atrophy of terminal follicles that normally produce thick scalp hair and transforms them into vellus-like follicles, eventually yielding fine, downy hair that is barely perceptible. Androgenetic alopecia is expressed in males as baldness of the vertex of the scalp and is commonly referred to as male pattern baldness. In females, androgenetic alopecia appears as diffuse hair loss or thinning of the frontoparietal areas. As alopecia progresses with age, hairs in these predisposed areas miniaturize and appear to change from terminal hairs to resemble vellus hairs. In addition, as androgenetic alopecia continues, the number of hairs in the active growth anagen phase decreases while there is an increase the number of hairs in the telogen phase.

Androgenetic alopecia, which is sometimes referred to as “common baldness” or “male pattern baldness,” independent of its causes, is the cutaneous aping of a particular zone (i.e., the scalp). Androgenetic alopecia can be defined, on one hand, as atrophy, sclerosis or miniaturization of the hair follicles. On the other hand, androgenetic alopecia can be defined as a progressive shortening of the average duration of the anagen stage, which results in vellus hair prior to complete disappearance.

Hair loss is an extremely common condition among healthy adult males, and also occurs frequently in adult females. In fact, some degree of alopecia on the vertex from puberty onwards is thought to be a universal phenomenon in both men and women. Alopecia is also frequently observed in both pre- and post-pubertal patients as a side-effect of anti-cancer chemotherapy.

Despite the widespread occurrence of androgenetic alopecia, the need for prevention and therapy still exists. The lack of a proven and effective treatment for androgenetic alopecia has caused many afflicted individuals to adopt the practice of wearing a wig or toupee. Another extreme measure used to combat androgenetic alopecia, hair transplant surgery, is not available as an option in many cases (i.e., following chemotherapy) and offers, at best, only a partial remedy. At the same, the latter treatment suffers from a number of disadvantages, including the need for surgery.

A common non-surgical treatment for stimulating hair growth is minoxidil, or Rogaine®. Minoxidil is a vasodilatory drug which has serious side-effects when administered orally for the treatment of hypertension. At the same time, topical application of minoxidil for the treatment of androgenetic alopecia is only partially effective and suffers from a number of disadvantages. For example, minoxidil is only recommended for treatment of male pattern alopecia of the vertex (i.e., frontal recession), has to be applied twice daily for at least four months, and requires a normal scalp with no local abrasions, dermatitis or sunburn—conditions that can increase absorption into the blood stream and the concomitant risk of side-effects. Further, minoxidil is of limited effectiveness. For example, there is no significant increase in terminal hair growth between minoxidil and placebo treatment groups after four months of treatment. In patients who do respond to minoxidil treatment, the new hair is likely to be shed within a few months after stopping treatment.

Androgens are responsible for many physiological functions in both males and females. Androgen action is mediated by specific intracellular hormone receptors expressed in androgen responsive cells. Testosterone, the major circulating androgen, is secreted by Leydig cells of the testes under the stimulation of pituitary-derived luteinizing hormone (LH). However, reduction of the 4, 5 double bond of testosterone to dihydrotestosterone (DHT) is required in some target tissues, such as prostate and skin, for androgen action. Steroid 5-alpha-reductases in target tissues catalyze conversion of testosterone to DHT.

The requirement for DHT to act as an agonist in these target tissues has been highlighted by studies of steroid 5-alpha-reductase in deficient individuals who have vestigial prostate glands and do not suffer from male pattern baldness. Thus, inhibition of the conversion of testosterone to DHT in these target tissues is anticipated to be useful in the treatment of a variety of androgen responsive diseases (i.e., benign prostatic hyperplasia, prostate cancer, acne, male pattern baldness and hirsutism).

Additionally, it has been discovered that two isozymes of 5-alpha-reductase exist in humans that differ in their tissue distribution, affinity for testosterone, pH profile and sensitivity to inhibitors. The steroid 5-alpha-reductase deficient individuals are deficient in the type 2,5-alpha-reductase enzyme, which is the predominant isozyme present in the prostate, while the type 1 isozyme is predominant in the skin. The relative value of isozyme specific and dual inhibitors of the two isozymes of 5-alpha-reductase will depend upon the type of disease treated (benign prostatic hyperplasia, prostate cancer, acne, male pattern baldness or hirsutism) as well as the stage of the disease (prevention versus treatment) and the anticipated side-effects in the intended patients (for example treatment of acne vulgaris in pubescent males).

Because of their valuable therapeutic potential, testosterone 5-alpha-reductase inhibitors have been the subject of active research worldwide. For example, finasteride, or Proscar®, i.e., is an inhibitor of 5-alpha-reductase-2 and is known to be useful for the treatment of hyper-androgenetic conditions.

Androgens are the most obvious regulators of human hair growth in both sexes. Androgens have paradoxically contrasting effects on follicles depending on their location in the body. Androgens stimulate hair growth in many locations (i.e., beard, axilla) while inhibiting scalp hair growth in genetically predisposed individuals. Androgens act on the hair follicles via the dermal papilla, presumably by altering the production of regulatory factors effecting the dermal papilla cells. Cultured dermal papilla cells secrete soluble, proteinaceous factors which are mitogenic for other dermal papilla cells, outer root sheath cells, epidermal keratinocytes and endothelial cells. Androgen sensitive cells from beard or balding scalp reflect their in vivo androgenetic responses by responding to testosterone, by increasing (i.e., beard) or decreasing (i.e., balding) their mitogenic ability.

Herbal formulations always have attracted considerable attention because of their good activity, relatively low toxicity, and comparatively lesser or non-existent side effects with synthetic drugs. One such herbal formulation, amla oil, comes from the Indian gooseberry (Phyllanthus emblica), which is a fruit that comes from the amalaki tree that is native to India. Amla contains plentiful vitamin C, minerals, and antioxidants. Research suggests that amla is excellent for boosting hair health and increasing hair growth. For instance, some research indicates that amla is effective at keeping hair parasites away because it is the most toxic to hair parasites, thereby improving hair appearance, and increasing hair growth. Moreover, in another study, researchers looked at the ability of amla and other herbal hair oils, such as coconut oil, to improve hair health and boost growth in animals. The researchers placed patches containing amla oil in various concentrations on the animals' fur and monitored fur growth for 15 days. They found amla oil to cause “significant” and rather rapid fur growth, and made fur healthier with no toxicity. Further, researchers have determined that the ideal concentration of amla oil to be a 7.5 percent solution with water for promoting hair growth.

Coconut oil is predominantly composed of saturated fatty acids, such as Lauric acid, along with a few medium, chain fatty acid. The saturated fat possesses antioxidants and anti-aging properties by countering the effects of aging, whitening of hair, and sagging of skin. Additionally, upon reaction with certain enzymes within the body, the acids can convert themselves into antimicrobial agents such as monocaprin and monolaurin. These chemical derivatives protect the body both internally and externally from conditions such as rashes, ringworm, and dermatitis. Coconut oil also possesses a significant amount of moisture retaining capacity, making the oil ideal for skin care use. Another primary constituent of coconut oil is Vitamin E, which helps keep hair and skin healthy and keeps individual organic system functioning smoothly. These characteristics make coconut oil idea for the treatment of androgenetic alopecia.

Therefore, there is a need in the art for a hair growth composition including a combination of hair growth promoting agents such as finasteride, minoxidil, amla, and coconut oil for providing an improved composition that promotes hair growth and resists androgenetic alopecia.

### SUMMARY OF THE INVENTION

In view of the foregoing disadvantages inherent in the known types of hair growth compositions now present in the prior art, the present invention provides a new and improved hair growth composition wherein the same can be utilized for providing convenience for the user when treating and preventing androgenetic alopecia.

In one embodiment of the present invention, the hair growth composition comprises 2% to 10% weight/volume (w/v) of minoxidil, 0.1% to 0.5% w/v of finasteride, 7% to 8% w/v of amla oil, and 1% to 8% w/v of coconut oil. The compositions is configured to treat hair loss by promoting and inducing hair growth/regrowth of hair in individuals suffering from androgenetic alopecia, or “common baldness.

In another embodiment of the present invention, the hair growth composition comprises 5% w/v of minoxidil, 0.1% w/v of finasteride, 7.5% amla oil, and 4.5% coconut oil. This specific concentration of ingredients is configured to provide the most synergistic, highly efficacious, and rapid effect, inducing the maximum amount of hair growth activity in individuals suffering from androgenetic alopecia.

### DETAILED DESCRIPTION OF THE INVENTION

Reference is made herein to the attached drawings. Like reference numerals are used throughout the drawings to depict like or similar elements of the hair growth composition. The figures are intended for representative purposes only and should not be considered to be limiting in any respect.

The present invention provides a novel hair growth composition configured to treat hair loss by promoting and inducing hair growth and regrowth in individuals suffering from androgenetic alopecia, or “common baldness.” The composition includes a mixture of minoxidil, finasteride, amla oil, and coconut oil. The combination of these ingredients provides an improved composition that synergistically treats androgenetic alopecia.

The hair growth composition comprises 2% to 10% weight/volume (w/v) of minoxidil, 0.1% to 0.5% w/v of finasteride, 7% to 8% w/v of amla oil, and 1% to 8% w/v of coconut oil in a 100 mL aqueous solution or solvent. The hair growth composition preferably comprises 5% w/v of minoxidil, 0.1% w/v of finasteride, 7.5% amla oil, and 4.5% coconut oil in a 100 mL aqueous solution. In other terms, the hair growth composition preferably comprises 5 g/100 mL of minoxidil, 1 mg/100 mL of finasteride, 7.5 g/mL of amla oil, and 4.5 g/100 mL of coconut oil. This specific combination of ingredients is configured to provide the maximum amount of hair growth activity in an individual.

The ingredients of the hair growth composition are dissolved in a 100 mL solvent or water mixture, capable of dissolving and retaining the ingredients therein and allowing the ingredients to reach the skin and hair follicle. The hair growth composition may be in the form of a gel, paste, cream, lotion, or ointment, or on a carrier (e.g. on sponges, in dispensers or cotton applicators).

In yet another embodiment of the present invention, one dose of the hair growth composition comprises 40 mg-200 mg of minoxidil, 0.2 mg-1 mg of finasteride, 70 mg-80 mg of amla oil, and 1000 mg-14,000 mg of coconut oil. In a preferred dose, the hair growth composition comprises 100 mg of minoxidil, 0.1 mg of finasteride, 75 mg of amla oil, and 1000 mg of coconut oil. This preferred dose is configured to provide the maximum amount of hair growth activity in an individual. It is contemplated that the above dose can be administered via pill form, capsule form, or topical agent.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. 1) A hair growth composition, comprising:
2%-10% w/v of minoxidil;
0.1%-0.5% w/v of finasteride;
7%-8% w/v of amla oil; and
1%-8% w/v of coconut oil.

2) The hair growth composition of claim 1, comprising 5% w/v of minoxidil.

3) The hair growth composition of claim 1, comprising 0.1% w/v of finasteride.

4) The hair growth composition of claim 1, comprising 7.5% w/v of amla oil.

5) The hair growth composition of claim 2, comprising 0.1% w/v of finasteride.

6) The hair growth composition of claim 6, comprising 7.5% w/v of amla oil.

7) A hair growth composition, consisting essentially of:
2%-10% w/v of minoxidil;
0.1%-0.5% w/v of finasteride;
7%-8% w/v of amla oil; and
1%-8% w/v of coconut oil.

8) The hair growth composition of claim 7, consisting essentially of 5% w/v of minoxidil.

9) The hair growth composition of claim 7, consisting essentially of 0.1% w/v of finasteride.

10) The hair growth composition of claim 7, consisting essentially of 7.5% w/v of amla oil.

11) The hair growth composition of claim 8, consisting essentially of 0.1% w/v of finasteride.

12) The hair growth composition of claim 11, consisting essentially 7.5% w/v of amla oil.

13) A hair growth composition, consisting essentially of:
40 mg-200 mg of minoxidil;
0.2 mg-1 mg of finasteride;
70 mg-80 mg of amla oil; and
1000 mg-14,000 mg of coconut oil.

14) The hair growth composition of claim 13, consisting essentially of 100 mg minoxidil.

15) The hair growth composition of claim 13, consisting essentially off 1 mg of finasteride.

16) The hair growth composition of claim 13, consisting essential of 75 mg of amla oil.

17) The hair growth composition of claim 14, consisting essentially of 1 mg of finasteride.

18) The hair growth composition of claim 17, consisting essentially of 75 mg of amla oil.
